(19) United States
(12) Patent Application Publication
(10) Pub. No.: US 2019/0187089 A1
(43) Pub. Date: Jun. 20, 2019

(54) METHOD FOR OPERATING AN AMPEROMETRIC SENSOR, AMPEROMETRIC SENSOR, AND METHOD FOR MONITORING A MEASURING FLUID IN A FLUID LINE NETWORK

(71) Applicant: Endress+Hauser Conducta GmbH+Co. KG, Gerlingen (DE)

(72) Inventors: Lars Bondzio, Groß-Umstadt (DE); Erik Hennings, Freiberg (DE); Stephan Jugert, Dresden (DE); Thomas Pfauch, Leipzig (DE); Jens Vettermann, Großweitzschen (DE)

(21) Appl. No.: 16/220,227

(22) Filed: Dec. 14, 2018

(30) Foreign Application Priority Data
Dec. 14, 2017 (DE) 10 2017 129 979.3

(51) Int. Cl. G01N 27/30; G01N 33/18

(52) U.S. Cl. CPC G01N 27/302; G01N 33/18; G01N 27/304; G01N 27/301

(57) ABSTRACT

The present disclosure relates to a method for operating an amperometric sensor for detecting measured values of at least one first measurand that represents a concentration of a first analyte in a measuring fluid, and of at least one second measurand which represents a concentration of a second analyte different from the first analyte. The method includes steps of detecting measured values of the first measurand in a first operating mode of the amperometric sensor, switching the amperometric sensor to a second operating mode different from the first operating mode, and detecting measured values of the second measurand in the second operating mode of the amperometric sensor. The method can be used in monitoring the water quality in drinking water installations or drinking water networks.

### CROSS-REFERENCE TO RELATED APPLICATION

The present application is related to and claims the priority benefit of German Patent Application No. 10 2017 129 979.3, filed on Dec. 14, 2017, the entire contents of which are incorporated herein by reference.

### TECHNICAL FIELD

The present disclosure relates to a method for operating an amperometric sensor for detecting measured values of at least one first measurand which represents a concentration of a first analyte in a measuring medium, and a second measurand which represents a concentration of a second analyte different from the first analyte, and an amperometric sensor suitable for carrying out the method. The present disclosure further relates to a method for monitoring a measuring fluid in a fluid line network.

### BACKGROUND

Amperometric sensors generally comprise a measuring probe which can be immersed in a measuring fluid and can be connected to superordinate sensor electronics, e.g., a measuring transducer or other electronics for measured value processing. The measurement probe of these sensors comprises a probe housing in which is formed a housing chamber sealed by a membrane and filled with an inner electrolyte. The membrane is arranged within a region of the measurement probe designated for contact with the measuring fluid and is permeable to an analyte, such as a gas, so that the analyte can pass from the measuring fluid into the housing chamber or vice versa.

At least two electrodes contacting the inner electrolyte are arranged within the housing chamber. In some embodiments, three electrodes may also be present. The electrodes are connected in an electrically conductive manner to a probe circuit that is arranged outside of the housing chamber and can be a component of on-site electronics arranged in the probe housing, for example. One of the electrodes is used as a working electrode, another as a counter electrode. For determining the measurand, a voltage is applied by means of the measuring circuit between the working electrode and the counter electrode at a level such that any analyte present in the inner electrolyte is electrochemically converted at the working electrode. The current flowing through the working electrode and the electrolyte in the process, such as between the working electrode and counter electrode, is detected by means of the measuring circuit as a measure of the concentration of the analyte in the measuring fluid. The analyte concentration in the measuring fluid can, for example, be indicated as a partial pressure for a measuring gas and as a concentration for a measuring liquid. In applications with three electrodes, the potential of the working electrode or the current flow through the working electrode may be regulated by means of a third reference electrode through which current does not flow.

Amperometric sensors are used in analytical engineering, process metrology and environmental metrology for monitoring concentrations of specific analytes in measuring media, e.g., process media, products of synthesis methods or preparation methods, or in the environment, e.g., to monitor concentrations of specific analytes in bodies of water. An example of such a use of amperometric sensors is monitoring the quality of measuring fluids in lines of a fluid line network. If fluids from different supply sources are fed into a fluid line network, they can have different constituents. To monitor the various measuring fluids transported within the fluid line network, a number of sensors must be provided in the measuring fluid corresponding to the number of measuring fluid constituents to be monitored. The associated problems will be presented in the following as examples with reference to a drinking water network. They can be transferred analogously to other fluid line networks.

A drinking water network frequently receives drinking water from various supply sources, for example from various water suppliers which obtain water from wells, groundwater, open bodies of water, such as lakes or rivers, or else in treated form from water treatment plants in which water is purified, desalinated, sterilized and optionally mineralized in order to be able to be used as drinking water. The supply sources are connected to a supply system which can simultaneously also serve as a water treatment plant and supplies drinking water to the drinking water network. Frequently, during an initial time period which can be several hours of the day, the supply system feeds water taken from a first supply source into the drinking water network. After this first time period has elapsed, the supply system changes the supply source and feeds water taken from the second supply source into the drinking water network during a second time period, which can likewise be several hours. In this way, the supply system can alternatingly supply water from the two supply sources. Of course, it can also use more than two supply sources and supply water sequentially from one of the plurality of supply sources.

Drinking water is frequently sterilized in supply or water treatment plants and, therefore, when it is fed into the drinking water network, generally still contains the sterilizing agent or reaction products of the sterilizing agent used for disinfection. Chlorine dioxide or ozone, for example, are used as sterilizing agents for water or drinking water. The concentration thereof in the water provides information on the achieved disinfecting effect. Chlorine and bromine are also used as a sterilizing agent. They are present in water in a pH-dependent equilibrium as dissolved chlorine (Cl₂) or as dissolved bromine (Cl₂), hypochlorous acid HOCl or hypobromous acid HOBr, and as hypochlorite or hypobromite OCl— or hypobromite OBr—. In order to provide information on the achieved disinfecting effect when sterilizing with chlorine or bromine, the parameters “free chlorine” or “free bromine” can be determined which correspond to the amount of hypochlorous acid or hypobromous acid in the water. The concentrations of said sterilizing agent or its reaction products can also be used to determine and monitor amperometric measurements.

Frequently, the composition of the water from a first supply source differs from the composition of the water from a second supply source. For example, the dissolved ions or gases contained in the water can vary in their concentrations. In addition, the water of the first supply source can be sterilized by means of a first method, for example using chlorine, while the water of the second supply source can be sterilized by means of a different second method, e.g., using chlorine dioxide. In this case, the water of the first source contains hypochlorous acid but is free of chlorine dioxide, while the water of the second source does not contain any hypochlorous acid, but instead contains a certain amount of chlorine dioxide.

In order to monitor the concentration of the sterilizing agent or its reaction products in water in a water network, for example a drinking water network, amperometric sensors, also referred to as disinfection sensors, are used today. Each of these sensors is configured to monitor the concentration of a single sterilizing agent or its reaction product. If the drinking water network is supplied from a plurality of supply sources which differ with regard to the sterilizing agent used for disinfection, a specially designed amperometric sensor is provided for each of the sterilizing agents used for monitoring the drinking water in the drinking water network. This requires the installation of a plurality of individual sensors. In many cases, fluid is withdrawn from a line of the line network via a bypass line or a sampling line and fed as a sample to a sensor installed in a bypass line or in a flow cell connected to the sampling line. In so doing, filters or other means for sample preparation before supplying the sample to the sensor can also be provided. Given a plurality of individual sensors, these additional lines and means for sample preparation multiply accordingly. In addition to this high installation effort, a high maintenance effort also results, since each sensor has to be regularly calibrated, and wearing parts have to be exchanged. All this is also associated with correspondingly high costs.

It is conceivable to provide the individual sensors together, at least in a common housing. This housing can, for example, have two chambers, in each of which an inner electrolyte, as well as a working electrode and counter electrode pair are arranged. The chambers can be closed by a common membrane or by a single membrane in each case. If a common membrane is used, it must be designed such that it is permeable to all analytes to be detected. This can be ensured, for example, by a plurality of pores with a size distribution that is adapted to the analyte to be detected and/or by selecting a suitable membrane material. In this case, the inner electrolytes and the voltages applied between the working electrode and counter electrode are optimally selected for the quantitative determination of a single analyte, for example for determining one of the aforementioned sterilizing agents. The sensors combined in this way in a common housing can have a common probe circuit and common, superordinate sensor electronics. These can be configured to cyclically or periodically successively detect measured values from each sensor. In this way, part of the installation effort is avoided and common electronics, and thus also a single operating device, can be used. This at least facilitates operation and maintenance and reduces the space requirement. Because of the smaller space requirement of such a combined device in comparison with a plurality of individual sensors, it is also conceivable to install such a combined device directly in a fluid line of the fluid line network.

As mentioned above, the technical challenges described here with water monitoring in drinking water networks also occur in a very analogous manner in various other applications in which a plurality of constituents, possibly different measuring fluids, are monitored in a fluid line network.

### SUMMARY

It is, therefore, the object of the present disclosure to further reduce the effort for monitoring a plurality of analytes by means of amperometric sensors, in particular in networks which are fed with measuring fluids from different supply sources and, therefore, contain different constituents and thus different analytes to be monitored.

A method is provided, according to the present disclosure, for operating an amperometric sensor for detecting measured values of at least one first measurand that represents a concentration of a first analyte in a measuring fluid, and of at least one second measurand which represents a concentration of a second analyte different from the first analyte. The method includes steps of detecting measured values of the first measurand in a first operating mode of the amperometric sensor, switching the amperometric sensor to a second operating mode different from the first operating mode, and detecting measured values of the second measurand in a second operating mode of the amperometric sensor.

By switching the amperometric sensor from a first to a second operating mode, and thus detecting measured values of the concentration of the first analyte while in the first operating mode and detecting measured values of the concentration of the second analyte while in the second operating mode, it is possible to monitor the concentration of two different analytes using one and the same sensor. The installation and operating effort is thus considerably reduced compared with the above-described solutions known from the prior art.

A measurand that represents the concentration of an analyte can be, in addition to the concentration of the analyte itself, an activity of the analyte, a partial pressure of the analyte, a mass or volume fraction of the analyte in the measuring fluid, or some other measurand associated with the analyte concentration in the measuring fluid.

The detection of measured values of the first measurand in the first operating mode can include a step of applying a predetermined first voltage between a working electrode of the amperometric sensor and a counter electrode of the amperometric sensor, wherein the working electrode and the counter electrode contact an inner electrolyte separated from the measuring fluid via a membrane permeable to the first and second analytes. The detection of measured values of the first measurand can also include generating measurement signals representative of a current passing through the inner electrolyte at the predetermined first voltage across the working electrode, and determining the measured values of the first measurand using a first evaluation process from the measurement signals.

The detection of measured values of the second measurand in a second operating mode can include a step of applying a predetermined second voltage, different from the first voltage, between the working electrode and the counter electrode of the amperometric sensor. The detection of measured values of the second measurand also includes steps of generating measurement signals representative of a current passing through the inner electrolyte at the predetermined second voltage across the working electrode, and determining the measured values of the second measurand from the measurement signals using a second evaluation method different from the first evaluation method.

The first evaluation method can, for example, comprise the determination of measured values on the basis of a first function, determined in particular by a calibration which associates values of the measurement signals with values of the first measurand. The second evaluation method may comprise the determination of measured values on the basis of a second function, determined in particular by a calibration, which associates values of the second measurand to values of the measurement signals. The two functions can differ from one another.

The first and/or the second evaluation method can provide for at least one additionally detected measured value of at least one auxiliary measurand, for example a pH and/or temperature measured value, to be taken into account in the determination of the measured values, for example in the form of a temperature or pH compensation of the measured value of the first or second measurand determined on the basis of the first or second function. The auxiliary measurand can be made available by a further sensor, for example a temperature sensor or a pH sensor, which is arranged in the vicinity of the amperometric sensor or integrated in the amperometric sensor such that the value of the auxiliary measurand detected thereby basically corresponds to the value of the auxiliary measurand of the measured medium at the location of the membrane of the amperometric sensor.

The switchover of the amperometric sensor to a second operating mode different from the first operating mode can be automatically carried out by a sensor circuit of the amperometric sensor after a predetermined time interval has elapsed, or triggered by user input, or triggered by a trigger signal received from the sensor circuit. The switchover can take place in such a way that the sensor circuit runs a first operating program before switching which serves to determine measured values on the basis of the first evaluation method and, after switching, runs a second operating program which serves to determine measured values on the basis of the second evaluation method.

In a manner very similar as described with reference to the first and second operating modes, the sensor can also be operated in more than two operating modes in order to determine more than two different measurands. For example, the sensor can be switched to at least one further operating mode in order to determine at least one further measurand that represents a concentration of an analyte different from the first and second analytes.

The first analyte and the second analyte can be constituents contained in the measuring fluid, for example water, for example sterilizing agents or reaction products of sterilizing agents. Suitable sterilizing agents are, for example, free chlorine, chlorine dioxide, free bromine, bromine dioxide and ozone. The first or second analyte can also be another amperometrically determinable gas, for example ammonia, carbon monoxide, carbon dioxide or oxygen.

The measuring fluid can be a gas or a liquid. The measuring fluid can be, for example, water, in particular drinking water or waste water.

The present disclosure also comprises an amperometric sensor for detecting measured values of at least one first measurand, which represents a concentration of a first analyte in a measuring medium, and at least one second measurand, which represents a concentration of a second analyte different from the first analyte. The amperometric sensor includes a sensor housing in which a housing chamber is formed, and a membrane sealing the housing chamber and being permeable to the first and second analytes. The amperometric sensor also includes a working electrode and a counter electrode, both of which are arranged within the housing chamber. The amperometric sensor further includes an inner electrolyte contained in the housing chamber and in contact with the membrane, the working electrode and the counter electrode, and a sensor circuit that is connected in an electrically conductive manner to the working and counter electrodes and is configured to apply a given voltage between the working electrode and the counter electrode and to detect measuring signals representing current flowing through the working electrode and electrolyte at the given voltage. The sensor circuit is additionally configured to be operated at least in a first operating mode in which it determines measured values of the first measurand from the measurement signals, and wherein the sensor circuit is configured to be switched from the first operating mode into at least one second operating mode and operated in the second operating mode in which it determines measured values of the second measurand from the measurement signals.

The sensor circuit can be divided into a probe circuit which is arranged in the sensor housing and an evaluation circuit which is superordinate to the probe circuit. In this case, the probe circuit and the evaluation circuit are connected to one another by wire or wirelessly for communication and for supplying energy to the probe circuit by the evaluation circuit. The probe circuit can, for example, be in the form of on-site electronics of the amperometric sensor which are accommodated in the housing intended for contacting the measuring fluid. The evaluation circuit can be configured in the form of electronics, for example a measuring transducer, which can be connected to the sensor housing via a cable connection. It can also be designed in the form of an operating device which can be wirelessly connected to the sensor, such as a smartphone or other smart device.

The sensor circuit can comprise one or a plurality of microprocessors, as well as memory elements, wherein operating programs are saved in the memory elements and can be run by the microprocessor or microprocessors for detecting measurement signals and for determining measured values of the measurands.

In the first operating mode, the sensor circuit can be configured to apply a given first voltage between the cathode and the counter electrode, and to determine, by means of a first evaluation method, measured values of the first measurand from the measurement signals detected while applying the given first voltage. In the second operating mode, the sensor circuit may be configured to apply a given second voltage, which is different from the first voltage, between the cathode and the counter electrode, and to determine, by means of a second evaluation method, measured values of the second measurand from the measurement signals detected while applying the second voltage.

The first and second evaluation methods can differ in the manner described above with reference to the method. The evaluation methods are implemented as operating programs executable by the microprocessor or microprocessors of the sensor circuit, and may be run by the evaluation circuit.

The sensor circuit can be configured to switch to and operate in at least one additional operating mode, in particular in a plurality of additional operating modes, in order to determine measured values of other measurands different from the first and second measurand.

The sensor circuit can comprise input means by means of which a user can switch the sensor from the first to the second operating mode. For example, the evaluation circuit designed as a measuring transducer or operating device can have a display on which a menu can be shown in which a user selects the first or second operating mode. For example, the first and second measurands can be displayed to the user. By selecting one of the measurands, the user can select the first or second operating mode. Thus, he does not have to know or specify how the first or second evaluation method is specifically designed, nor does he have to dictate a specific voltage to be applied between the working electrode and the counter electrode. The sensor circuit can independently establish selection of the voltage to be applied and/or the evaluation method based on the selection of the measurand.

The sensor circuit can comprise a signal input by means of which the sensor circuit can receive a trigger signal from a device connected for communication with the sensor circuit, wherein the sensor circuit is configured to switch the sensor to the first or the second operating mode on the basis of the trigger signal. The device connected for communicating with the sensor circuit can, for example, be a control unit which controls the supply of measuring fluid in a line in which the amperometric sensor is arranged for detecting measured values. In this way, the control unit can output a trigger signal to the sensor so that it switches from the first operation mode to the second operation mode when it changes the measuring fluid, e.g., feeds measuring fluid from a second supply source into the line instead of measuring fluid from the first supply source.

The sensor circuit can alternatively or additionally include a time switch (timer) which, after expiration of a predetermined period of time, is configured to switch the sensor from the first operating mode into the second operating mode.

The present disclosure also relates to a method for monitoring measuring fluids in a fluid line network by means of the amperometric sensor disclosed herein. The method includes steps of installing the amperometric sensor in a container through which measuring fluid flows, such as a pipeline or a pipeline bypass, of the fluid line network, and detecting measured values of the first measurand in the first operating mode of the amperometric sensor. The method also includes steps of switching the amperometric sensor from the first operating mode to the second operating mode, and detecting measured values of the second measurand in the second operating mode of the amperometric sensor.

Switching can occur after a predetermined period of time or at a given point in time. As described above, this can be triggered by a user input, by a trigger signal, or by the expiration of a period of time monitored by a timer on the basis of a signal of the timer.

The network can be connected to a first supply source and to a second supply source, wherein a controller exclusively feeds measuring fluid into the network from the first supply source over a first period of time, and exclusively feeds measuring fluid of the second supply source over a subsequent, second period of time, and wherein the controller feeds a trigger signal to the amperometric sensor, and wherein the switching of the amperometric sensor from the first operating mode to the second operating mode is triggered by the trigger signal. The measuring fluid from the first supply source can have other constituents than the measuring fluid from the second supply source. Thus, the first measurand is dependent on the concentration of a first constituent which is contained, for example, in the measuring fluid of the first supply source, while the second measurand depends on the concentration of a second constituent which is different from the first constituent and which is contained in the measuring fluid of the second supply source. The first or the second measurand can be measured by means of the sensor, depending on which measuring fluid is being fed into the fluid line network.

The measuring fluid can for example be water, e.g. waste water or drinking water. The water network can correspondingly be a drinking water supply network or also a waste water network. If the water network is a drinking water network, the first measurand can be, for example, a concentration of a first sterilizing agent in the water, while the second measurand can be a concentration of a second sterilizing agent in the water different from the first sterilizing agent.

A plurality of further embodiments and modifications to the methods described herein and sensor described herein are also conceivable. Although the operation of the sensor and methods have been illustrated herein with reference to determining and monitoring sterilizing agent concentrations and with reference to water and drinking water supply networks, it can in principle also be applied to determining other constituents to be monitored as analytes in fluids transported and monitored in networks. The sensor can also be operated in more than two operating modes in order to alternately or cyclically successively monitor the concentrations of more than two different analytes.

### BRIEF DESCRIPTION OF THE DRAWINGS

In the following, the present disclosure is described in more detail with reference to the exemplary embodiments shown in the figures. The figures show:

FIG. 1 shows a schematic representation of an amperometric sensor according to a first embodiment; and

FIG. 2 shows a schematic representation of a drinking water supply network.

### DETAILED DESCRIPTION

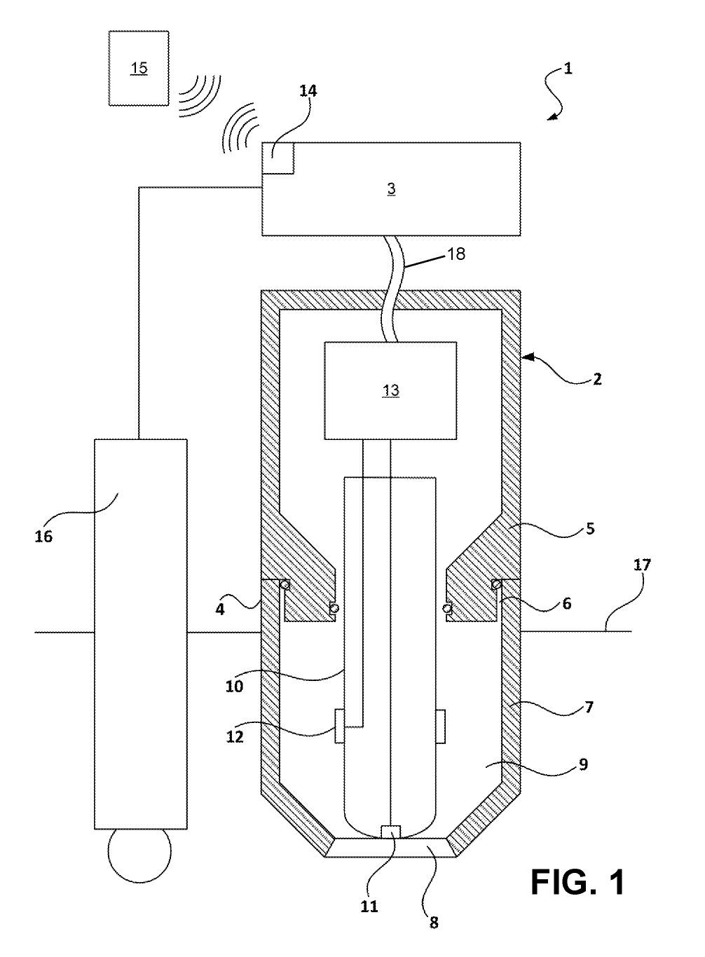

FIG. 1 schematically shows an amperometric sensor 1 for determining a concentration of different gaseous analytes in a measuring fluid. The measuring fluid can be a gas mixture containing the analyte, or a liquid in which the analyte is dissolved. The sensor 1 is arranged in a pipeline 17 conducting the measuring fluid such that its front end section is in contact with the measuring fluid flowing in the pipeline 17.

The sensor 1 comprises a substantially cylindrical measuring probe 2 with a probe circuit 13 contained in the measuring probe 2, and superordinate evaluation electronics 3 connected to the probe circuit 13 for communication. In the present example, the superordinate evaluation electronics 3 can be a measurement transmitter. The evaluation electronics 3 and the probe circuit 13 together form a sensor circuit whose functions can be suitably divided between the probe circuit 13 and the evaluation electronics 3.

The measurement probe 2 comprises a probe housing 4 that, in the example shown here, is made up of two parts, namely a probe body 5 and a sensor cap 7 that is connected by means of a screw connection 6 with the probe body 5 so as to be detachable. In the present example, the probe housing 4 consists of stainless steel but can also be formed from an electrically non-conductive material, for example a polymer material, such as polyether ether ketone (PEEK), polytetrafluoroethylene (PTFE) or polyvinylidene fluoride (PVDF). The sensor cap 7 has an essentially cylindrical cap base body that is sealed by a membrane 8 at its end facing away from the screw connection 6, which end is designated for immersion into the measuring fluid. This membrane 8 is firmly connected to the cap base body, for example via an integral connection, such as a glued or welded connection, or via a keyed connection, such as a clamped connection.

The sensor cap 7 and the probe body 5 include a housing chamber 9 that, in the present example, is filled with an aqueous electrolyte solution serving as inner electrolyte. On the back side, i.e., on its side facing away from the membrane 8, the housing chamber 9 is terminated in a liquid-tight manner by means of two seals so that the inner electrolyte does not get into the probe body 5 and also cannot exit out of the probe housing 4 via the screw connection 6.

The membrane 8 is formed from a plastic, for example silicone, PTFE or PVDF, and has a plurality of pores through which the analyte located in the measuring fluid can diffuse into the housing chamber 9. A diffusion in the reverse direction is also possible. In a state of equilibrium, the concentration of the satellite in the inner electrolyte thus correlates with an analyte constituent of the measuring fluid.

The measurement probe 2 further comprises a rod-shaped electrode body 10 that is attached at the rear side of the probe body 5 and whose forward segment facing toward the membrane 8 is arranged in the housing chamber 9. In the present example, the electrode body 10 consists of an electrically non-conductive material, for example a polymer material, such as PEEK, PTFE, or PVDF, or of glass. Embedded in the electrode body 10 is a first electrode that is referred to in the following as a working electrode 11 and that is exposed on the face of the electrode body 10 situated opposite the membrane 8 such that the working electrode 11 is in contact with the inner electrolyte. Otherwise, the electrode body 10 electrically insulates the working electrode from the inner electrolyte. The working electrode 11 may be formed from a noble metal, for example gold or platinum, at least at its exposed end. Moreover, on the electrode body 10, an annular or sleeve-shaped second electrode, referred to in the following as a counter electrode 12, is placed in a region that is wetted by the inner electrolyte. For example, this counter electrode 12 may be formed from silver provided with a silver chloride coating. Both the working electrode 11 and the counter electrode 12 are connected in an electrically conductive manner with the probe circuit 13 arranged within the probe body. The probe circuit 13 is designed to apply a predetermined voltage between the working electrode 11 and the counter electrode 12, which voltage is selected so that the analyte is electrochemically converted at the working electrode 11. The working electrode 11 rests on the membrane 8 such that only a thin film of the inner electrolyte forms between the working electrode 11 and the membrane 8. This contributes to a rapid response time of the sensor 1.

The sensor circuit includes a radio interface 14 by means of which it is connectable for communication to a superordinate unit 15, e.g., a process control or an operating device, e.g., a smartphone, a tablet PC or other smart device, or an independently movable operating device, e.g., a mobile or airborne vehicle drone. In the present example, the radio interface 14 is a component of the evaluation electronics 3; alternatively, however, it can also be part of the probe circuit 13.

The sensor circuit is designed to detect a current flowing through the inner electrolyte between the working electrode 11 and the counter electrode 12 given the applied voltage, and to generate and further process a measurement signal based thereupon. Since, as described further above, the analyte concentration that is present in the inner electrolyte is, in equilibrium, a measure of the analyte constituent of a measuring fluid in contact with the membrane 8, the measurement current flowing between working electrode 11 and counter electrode 12 is, for its part, a measure of the analyte constituent of the measuring fluid. Based on the measurement signal obtained, the sensor circuit can therefore determine and output a measured value of the analyte concentration or a related measurand in the measuring fluid from the measurement signal, optionally using a function previously determined by calibration.

The sensor 1 can be operated in at least two different operating modes. The sensor circuit is designed to be switched between the possible operating modes. Each operating mode serves to detect measured values of a concentration of a specific analyte in the measuring fluid. In that the sensor 1 can be switched between these different operating modes, it is capable of alternately monitoring a plurality of different analytes in a measuring fluid or in measuring fluids successively supplied to the sensor 1. This is explained in more detail below with reference to the example of monitoring the two analytes chlorine dioxide (ClO₂) and hypochlorous acid (HOCl), also referred to as free chlorine, in water as the measuring fluid.

In this case, the sensor 1 is designed to detect measured values of the concentration of hypochlorous acid in a first operating mode. It is further designed to detect measured values of the concentration of chlorine dioxide in the second operating mode. The membrane 8 is designed, in particular with regard to the diameters of its pores and with regard to its hydrophobicity, such that it is permeable both to hypochlorous acid and to chlorine dioxide. In the present example, the inner electrolyte is an aqueous electrolyte solution comprising a pH buffer. The pH buffer serves to keep the pH of the inner electrolyte stable in a pH range between 3 and 9. Advantageously, the pH of the inner electrolyte is in a neutral pH range around 7.

One or more operating programs, which can be run by a sensor circuit processor, are stored in a memory of the sensor circuit in order to operate the sensor in the first and second operating modes. The sensor circuit can switch the sensor to the first or the second operating mode on the basis of a trigger signal transmitted to the sensor circuit by the superordinate unit 15 which specifies the operating mode in which the sensor 1 is to be switched.

In the first operating mode, the sensor circuit applies a first specified voltage between the working electrode 11 and the counter electrode 12 with the working electrode 11 being connected as a cathode and the counter electrode 12 as an anode. The magnitude of the first voltage is such that hypochlorous acid at the working electrode 11 connected as a cathode is reduced to chloride according to the equation:

HOCl+H⁺2e⁻→Cl⁻+H₂O.

During the application of the first voltage, the sensor circuit detects the current flowing through the cathode and the electrolyte as a measurement signal and further processes it to determine a measured value of the concentration of hypochlorous acid in the measuring fluid. For this purpose, the sensor circuit digitizes the measurement signal or a processed measurement signal, e.g., a voltage value generated by a current-to-voltage converter from the current signal. From the digitized measurement signal, the sensor circuit determines a measured value of the hypochloride concentration by means of a first evaluation method implemented in the present example in an operating program which can be run by the sensor circuit. For this purpose, the sensor circuit can assign a value of the hypochloride concentration to the measurement signal, for example the digitized voltage, on the basis of a calibration function saved in a memory of the sensor circuit and determined by calibration. The sensor circuit can display the thus determined value via a display or via the remote interface to the superordinate unit 15.

As described above, hypochlorous acid is present in water in a pH-dependent equilibrium with hypochlorite. Since the calibration function saved in the sensor 1 was determined by means of a calibration at a particular pH, the measured value determined using this saved calibration function can be too high or too low when there are fluctuations in the pH in the measuring fluid. The evaluation method used in the present example therefore comprises a pH compensation of the determined measured value. For this purpose, the sensor circuit detects the pH measured values of a pH sensor 16 which is arranged in the pipeline 17 so that it is in contact with the measuring fluid for detecting the pH value. The additional pH sensor 16 can also be integrated in the measuring probe 2. In the present example, the sensor circuit is connected via a line 18 to on-site electronics of the pH sensor 16 so that it can detect pH measured values provided by the pH sensor 16. By means of the pH measured values, the sensor circuit performs a pH compensation of the measured values of the hypochlorous acid. In a very analogous manner, the sensor circuit can also be designed to perform a temperature compensation by means of temperature measured values determined by an additional sensor.

In the second operating mode, the sensor circuit applies a second specified voltage between the working electrode 11 and the counter electrode 12 with the working electrode 11 being connected as a cathode and the counter electrode 12 as an anode. The magnitude of the second voltage is such that chlorine dioxide at the working electrode connected as cathode 11 is reduced to chlorite according to the equation:

ClO₂+e⁻→ClO₂⁻.

During the application of the second voltage, the sensor circuit detects the current flowing through the cathode and the electrolyte provides a measuring signal, and further processes it to determine a measured value of the concentration of chlorine dioxide in the measuring fluid. As described above with regard to determining measured values of the concentration of hypochlorous acid, the sensor circuit digitizes the measurement signal and determines a measured value of the chlorine dioxide concentration from the digital values by means of a second evaluation method implemented in the present example in an operating program that can be run by the sensor circuit. For this purpose, the sensor circuit can assign values of the chlorine dioxide concentration to the digital values of the measurement signals on the basis of a calibration function saved in a memory of the sensor circuit and determined by calibration. The sensing circuit can output the values thus determined via a display or via a remote interface 14 to the superordinate unit 15. The sensor circuit can perform a temperature compensation of the measured values by means of an additional temperature sensor.

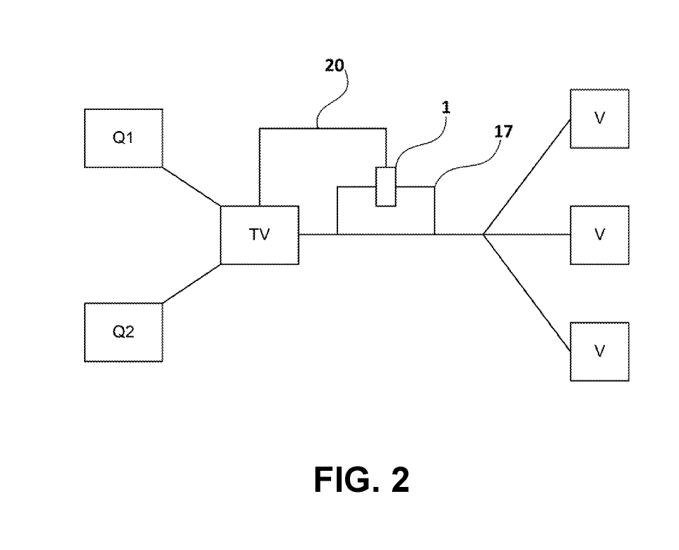

FIG. 2 schematically shows a water network, in the present example a drinking water network, which comprises a drinking water supply station TV. The drinking water supply station TV feeds water into the drinking water network which connects the consumers V to the drinking water supply station, and via which the consumers V are supplied with drinking water.

The drinking water supply station TV is connected to two supply sources Q1 and Q1. The first supply source Q1 is a water treatment plant in which water is purified and chlorine is added for disinfection. A second supply source Q2 is a water treatment plant in which water is added for disinfection of chlorine dioxide. In the example described here, the drinking water supply station TV receives water from the supply source Q1 during the day and water from the supply source Q2 during the night, wherein the drinking water supply station supplies drinking water from the second supply source Q2 to the water network starting at a certain time, such as 8 PM, and supplies water from the first supply source Q1 to the water network starting at a certain second time, such as 5 AM.

An amperometric sensor, such as the amperometric sensor 1 described with reference to FIG. 1, is arranged at one or more positions in the water network. The amperometric sensor 1 is arranged in a bypass line 17 in the example described here. It is connected via a line 20 to a controller of the drinking water supply station TV for communication.

During the day, i.e., while the drinking water supply station TV feeds water from the first supply source Q1 into the drinking water network, the amperometric sensor 1 is operated in the first operating mode and thus detects measured values of free chlorine in the water fed into the drinking water network. At the first time when the drinking water supply station TV feeds water from the second supply source Q2 into the drinking water network, it sends a triggering signal to the amperometric sensor 1, which thereupon switches to the second operating mode. From this time on, the amperometric sensor 1 detects measured values of the chlorine dioxide concentration in the water. If the drinking water supply station TV switches back to the first supply source Q1 at the second time, it sends a further trigger signal to the amperometric sensor 1, which thereafter switches back to the first operating mode in order to detect measured values of the free chlorine in the water. If, as in the present example, different polarization voltages are used in the first and in the second operating modes, the sensor 1 requires a certain polarization time, which is advantageously less than one hour, until it provides a stable measuring signal and thus reliable measured values.

In an alternative embodiment, it is also possible to dispense with the communication between the drinking water supply station TV and the amperometric sensor 1. In this case, the first and second times at which the supply source is switched can be saved in the sensor circuit of the amperometric sensor 1. The sensor circuit can comprise a time switch which switches the sensor 1 at the first time to the second operating mode and at the second time to the first operating mode.

In the above examples, the present disclosure was described with reference to the monitoring of the analytes chlorine dioxide and hypochlorous acid. Of course, the described amperometric sensor 1 and the corresponding methods can also be transferred quite analogously to other analytes, for example to other disinfection parameters. The drinking water network described with reference to FIG. 2 can be fed from additional supply sources; correspondingly, the amperometric sensor 1 can also comprise more than two operating modes to detect measured values of more than two different analytes. The switching between the individual operating modes can then, for example, be cyclical and/or adapted to the order in which the various supply sources are accessed.

1. A method for operating an amperometric sensor for detecting measured values of at least one first measurand that represents a concentration of a first analyte in a measuring fluid, and of at least one second measurand which represents a concentration of a second analyte different from the first analyte, wherein the method comprises:
detecting measured values of the first measurand in a first operating mode of the amperometric sensor;
switching the amperometric sensor to a second operating mode different from the first operating mode; and
detecting measured values of the second measurand in the second operating mode of the amperometric sensor.

2. The method of claim 1, wherein detecting measured values of the first measurand in the first operating mode comprises:
applying a predetermined first voltage between a working electrode of the amperometric sensor and a counter electrode of the amperometric sensor, wherein the working electrode and the counter electrode contact an inner electrolyte separated from the measuring fluid via a membrane permeable to the first and second analytes;
generating measurement signals representative of a current passing through the inner electrolyte at the predetermined first voltage across the working electrode; and
determining the measured values of the first measurand using a first evaluation process of the measurement signal.

3. The method of claim 2, wherein detecting measured values of the second measurand in a second operating mode comprises:
applying a predetermined second voltage different from the first voltage between the working electrode and the counter electrode of the amperometric sensor;
generating measurement signals representative of a current passing through the inner electrolyte at the predetermined second voltage across the working electrode; and
determining the measured values of the second measurand from the measurement signals using a second evaluation method different from the first evaluation method.

4. The method of claim 3, wherein the first evaluation method includes assigning values of the first measurand to the measurement signals based on a first calibration function determined by calibration.

5. The method of claim 3,
wherein the second evaluation method includes assigning values of the second measurand to the measurement signals based on a second calibration function determined by calibration.

6. The method of claim 5,
wherein the first and/or the second evaluation method additionally uses at least one measured value of at least one further auxiliary measurand to determine the measured values of the first or the second measurand.

7. The method of claim 6, wherein the auxiliary measurand includes a temperature or a pH.

8. The method of claim 1,
wherein the switchover of the amperometric sensor to the second operating mode different from the first operating mode is executed by a sensor circuit of the amperometric sensor after a predetermined time interval has elapsed, or triggered by user input, or triggered by a trigger signal received from the sensor circuit.

9. The method of claim 1, wherein the first analyte and the second analyte are selected from the group consisting of: hypochlorous acid, chlorine dioxide, hypobromous acid, bromine dioxide, ozone, oxygen, carbon monoxide, carbon dioxide, and ammonia.

10. An amperometric sensor for detecting measured values of at least one first measurand, which represents a concentration of a first analyte in a measuring medium, and at least one second measurand, which represents a concentration of a second analyte different from the first analyte, the amperometric sensor comprising:
a sensor housing in which a housing chamber is formed;
a membrane sealing the housing chamber and permeable to the first and second analytes;
a working electrode arranged within the housing chamber;
a counter electrode arranged within the housing chamber;
an inner electrolyte contained in the housing chamber and in contact with the membrane, the working electrode and the counter electrode; and
a sensor circuit electrically connected to the working electrode and the counter electrode and configured to apply a given voltage between the working electrode and the counter electrode and to detect measurement signals representing current flowing through the working electrode and the electrolyte at the given voltage;
wherein the sensor circuit is additionally configured to be operated in a first operating mode in which it determines measured values of the first measurand from the measurement signals, and wherein the sensor circuit is additionally configured to be switched from the first operating mode into a second operating mode in which the sensor circuit determines measured values of the second measurand from the measurement signals.

11. The amperometric sensor of claim 10, wherein, in the first operating mode, the sensor circuit is configured to apply a given first voltage between the working electrode and the counter electrode, and to determine, using a first evaluation method, measured values of the first measurand from the measurement signals detected while applying the given first voltage, and, in the second operating mode, to apply a given second voltage different from the first voltage between the working electrode and the counter electrode, and to determine, using a second evaluation method, measured values of the second measurand from the measurement signals detected while applying the given second voltage.

12. The amperometric sensor according of claim 10, wherein the sensor circuit can switch the sensor from the first operating mode into the second operating mode responsive to a user input.

13. The amperometric sensor of claim 10, wherein the sensor circuit is configured to receive a trigger signal from a device connected for communication with the sensor circuit, and wherein the sensor circuit is configured to switch the sensor to the first operating mode or the second operating mode based on the trigger signal.

14. The amperometric sensor of claim 10, wherein the sensor circuit includes a timer configured to switch the sensor from the first operating mode into the second operating mode after expiration of a predetermined period of time.

15. A method for monitoring a measuring fluid in a fluid line network using an amperometric sensor, comprising:
installing the amperometric sensor in a container through which the measuring fluid flows, detecting measured values of a first measurand in a first operating mode of the amperometric sensor;
switching the amperometric sensor from the first operating mode to a second operating mode; and
detecting measured values of the second measurand in the second operating mode of the amperometric sensor.

16. The method of claim 15, wherein the switching occurs after a predetermined period of time or at a predetermined time.

17. The method of claim 15,
wherein the fluid line network is connected to a first supply source and to a second supply source, and wherein a controller exclusively feeds measuring fluid into the network from the first supply source over a first period of time, and exclusively feeds measuring fluid of the second supply source over a second period of time, and wherein the controller feeds a trigger signal to the amperometric sensor, and wherein the switching of the amperometric sensor from the first operating mode to the second operating mode is triggered by the trigger signal.

18. The method of claim 15, wherein the fluid line network is a water network and the measuring fluid is water.
